(19) United States
(12) Patent Application Publication
(10) Pub. No.: US 2021/0100747 A1
(43) Pub. Date: Apr. 8, 2021

(54) Ground, Anti-radiation supplement

(71) Applicant: Tim Chrisman, Falls Church, VA (US)

(72) Inventor: Tim Chrisman, Falls Church, VA (US)

(21) Appl. No.: 17/122,185

(22) Filed: Dec. 15, 2020

(51) Int. Cl. A61K 9/48; A61K 36/258; A61K 36/9066; A61K 36/67; A61K 36/23; A61K 9/20

(52) U.S. Cl. CPC A61K 9/48; A61K 36/258; A61K 9/20; A61K 36/67; A61K 36/23; A61K 36/9066

(57) ABSTRACT

Ground is a nutritional supplement designed to slow the negative effects of radiation on the body. It combines 4 natural compounds shown in clinical trials to reduce radiation-induced inflammation, into a single supplement.

### GROUND COMPOSITION AND USE

### Formulation

Ingredients are combined into two tablets
1,000 mg Panax Ginseng
   Standardized at 80% Ginsenosides
500 mg Nigella sativa
500 mg Turmeric (Rhizomes) Curcumin C3 Complex
   Standardized to 95% Curcuminoids
   Also known as: Curcuma longa
435 mg Capsicum annuum L (Fruit)
   Extracted at 90,000 H.U.

Supplement is to be take two times daily preferably with a meal to help increase the body's resistance to radiation

1. A tablet or capsule consisting essentially of not less than 450 mg and not more than 550 mg Panax Ginseng standardized at 80% Ginsenosides, not less than 225 mg and not more than 275 mg Black Cumin Seed Oil (also known as Nigella Sativa), not less than 225 mg and not more than 275 mg Turmeric Rhizomes Curcumin C3 Complex (also known as Curcuna longa) standardized to 95% Curcuminoids, 200 mg and not more than 225 mg Capsicum Annuum L extracted to 90,000 Heat Units and not less than 8 mg and not more than 12 mg Bioperine© Black Pepper.
